(19) United States
(12) Patent Application Publication
(10) Pub. No.: US 2016/0287508 A1
(43) Pub. Date: Oct. 6, 2016

(54) Hair conditioning composition

(71) Applicant: RHODIA OPERATIONS, Paris (FR)

(72) Inventors: Hai Zhou ZHANG, Singapore (SG); Xi CHEN, Singapore (SG); William ZHAN, Singapore (SG)

(21) Appl. No.: 15/037,885

(22) PCT Filed: Nov. 20, 2014
(86) PCT No.: PCT/EP2014/075073

§ 371 Date: May 19, 2016

(30) Foreign Application Priority Data
Nov. 20, 2013 (EP) PCT/EP2013/074308

(51) Int. Cl. A61K 8/73; A61K 8/37; A61K 8/86; A61K 8/34; A61Q 5/12; A61K 8/44

(52) U.S. Cl. CPC A61K 8/737; A61Q 5/12; A61K 8/442; A61K 8/44; A61K 8/86; A61K 8/342; A61K 8/37; A61K 2800/436; A61K 2800/5426; A61K 2800/30

(57) ABSTRACT

The present invention concerns a hair conditioning composition comprising at least a fatty amphoteric surfactant, a fatty carboxylic acid and/or a fatty alcohol; and a cationic polysaccharide or cationic polysaccharide derivative, which can provide a good pearlescent effect and hair conditioning effect.

This application claims priority to PCT international application no. PCT/EP2013/074308 filed on Nov. 20, 2013, the whole content of this application being incorporated herein by reference for all purposes.

The present invention concerns a hair conditioning composition comprising at least a fatty amphoteric surfactant, a fatty carboxylic acid and/or a fatty alcohol; and a cationic polysaccharide or cationic polysaccharide derivative, which can provide a good pearlescent effect and hair conditioning effect.

### PRIOR ART

The following discussion of the prior art is provided to place the invention in an appropriate technical context and enable the advantages of it to be more fully understood. It should be appreciated, however, that any discussion of the prior art throughout the specification should not be considered as an express or implied admission that such prior art is widely known or forms part of common general knowledge in the field.

Human hair requires cleaning, since the hair fibers become soiled, both from environmental contamination as well as contamination and soiling from chemical agents produced by the body. Generally, shampooing is employed to clean the hair by removing excess soil and body oils which have built up on the hair fibers. Unfortunately, while being capable of cleaning the hair fibers, shampoos generally leave the hair stripped, over-processed and difficult to manage.

Hair conditioners deliver a number of desirable characteristics to hair upon treatment, including an improved wet or dry feel and a perception of softness. However, in order to secure high consumer acceptance of any hair conditioner composition, it is essential to provide consumer-desirable product aesthetics, for example not only an appealing neat product odor and a pleasant product color, but especially an appropriate product rheology and satisfactory physical product stability.

General hair conditioner systems are based on quaternary ammonium compounds, also named as quats, notably cetrimonium chloride, behentrimonium chloride, N,N-bis(stearoyl-oxy-ethyl) N,N-dimethyl ammonium chloride, N,N-bis(tallowoyl-oxy-ethyl) N,N-dimethyl ammonium chloride, N,N-bis(stearoyl-oxy-ethyl) N-(2-hydroxyethyl) N-methyl ammonium methylsulfate or 1,2-di(stearoyl-oxy)-3-trimethyl ammoniumpropane chloride.

However, quats provide a rather low water solubility, are known as very difficult to be biodegradable and provide confirmed eco toxicity, and it exists a general trend for this industry is to switch to other conditioning systems. Ester quats is one of the options, which provide better biodegradability and better eco toxicity. But even ester quats provides still some disadvantages as a not so long term stability in the final product due to a degradability that imposes to keep a very low pH in order to make it more stable. This so low pH value is not good for personal care.

Besides the conditioning effect, aesthetic aspects are also very important for personal care products. So a unique appearance like pearlescent effect is always welcomed by the consumers. But from the common quats based system, it is difficult to achieve a pearlescent appearance. Although, conventionally, some base materials giving a pearly luster has been used. As a main component for giving a pearly luster in the pearly luster composition, fatty acid glycol esters, fatty acid monoalkylolamides, fatty acids, and the like have been known. Among them, various fatty acid glycol esters have been studied as a main component in the pearly luster composition. However, these materials will not give any pearlescent effect in quats based conditioner system.

### INVENTION

The present invention is based on the surprising discovery that it is possible to obtain a stable hair conditioner composition with a shiny pearlescent effect as well that performs well on hair fibers, which comprises at least a fatty amphoteric surfactant, a fatty carboxylic acid and/or a fatty alcohol, and a cationic polysaccharide or cationic polysaccharide derivative; notably without the drawbacks generally associated with the use of quat materials.

These compounds indeed appear to be sufficiently efficient and notably same efficient in term of wet and dry softness, in comparison with the compounds classically used in the softening compositions such as cetrimonium chloride (CTAC), behentrimonium chloride (BTAC), di(palmiticcarboxyethyl) hydroxyethyl methyl ammonium methylsulfate (TEP), and dimethyl di(hydrogenated tallow) ammonium chloride (DHT).

Hair conditioning compositions of the present invention provide several benefits including include soft hair feel (wet and dry), ease of hair combing (wet and dry), hair detangling benefits (wet and dry), anti-static benefits, and hair manageability benefits. These compounds also provide the advantages to be more stable over time and different pH range, and provide a good compatibility with all other surfactants system.

The present invention concerns then a hair conditioning and pearlescent composition comprising at least:

a) 1-10% wt of an amphoteric surfactant carrying a C₁₂-C₂₂-alkyl or C₁₂-C₂₂-alkenyl;
b) 0.1-5% wt of an alcohol compound carrying a C₁₀-C₂₂-alkyl or C₁₀-C₂₂-alkenyl and/or a carboxylic acid compound carrying a C₁₀-C₂₂-alkyl or C₁₀-C₂₂-alkenyl; weight ratio of compound a) to compound b) is comprised between 1:2 and 20:1;
c) 0.01-2 wt of a cationic polysaccharide or cationic polysaccharide derivative; weight ratio of compound a) to compound c) is comprised between 3:1 and 50:1;
d) water;
percent by weight expressed in relation with the total weight of the composition;
wherein said composition comprises less than 0.5 wt % of quaternary ammonium salts, except compounds a), b) and c).

The present invention also concerns a hair conditioning and pearlescent composition comprising at least:

a) 1-10% wt of an amphoteric surfactant carrying a C₁₆-C₂₀-alkyl or C₁₆-C₂₀-alkenyl;
b) 0.1-5% wt of an alcohol compound carrying a C₁₀-C₂₂-alkyl or C₁₀-C₂₂-alkenyl and/or a carboxylic acid compound carrying a C₁₀-C₂₂-alkyl or C₁₀-C₂₂-alkenyl; weight ratio of compound a) to compound b) is comprised between 1:2 and 20:1;
c) 0.01-2% wt of a cationic polysaccharide or cationic polysaccharide derivative; weight ratio of compound a) to compound c) is comprised between 3:1 and 50:1;
d) water;
percent by weight expressed in relation with the total weight of the composition;
wherein said composition comprises less than 0.5 wt % of quaternary ammonium salts, except compounds a), b) and c).

The present invention also concerns a hair conditioning and pearlescent composition comprising at least:

a) 1-10% wt of an amphoteric surfactant carrying a C₁₆-C₂₀-alkyl or C₁₆-C₂₀-alkenyl;
b) 0.1-5% wt of an alcohol compound carrying a C₁₀-C₂₂-alkyl or C₁₀-C₂₂-alkenyl and/or a carboxylic acid compound carrying a C₁₀-C₂₂-alkyl or C₁₀-C₂₂-alkenyl; weight ratio of compound a) to compound b) is comprised between 1:2 and 20:1;
c) 0.01-2% wt of a cationic polysaccharide or cationic polysaccharide derivative; weight ratio of compound a) to compound c) is comprised between 3:1 and 50:1;
d) water;
percent by weight expressed in relation with the total weight of the composition;
wherein said composition comprises less than 0.5 wt % of quaternary ammonium salts, except compounds a), b) and c); said composition is substantially free or completely free of anionic agent.

The present invention also concerns a hair conditioning and pearlescent system consisting of:

a) an amphoteric surfactant carrying a C₁₂-C₂₂-alkyl or C₁₂-C₂₂-alkenyl;
b) an alcohol compound carrying a C₁₀-C₂₂-alkyl or C₁₀-C₂₂-alkenyl and/or a carboxylic acid compound carrying a C₁₀-C₂₂-alkyl or C₁₀-C₂₂-alkenyl; weight ratio of compound a) to compound b) is comprised between 1:2 and 20:1;
c) a cationic polysaccharide or cationic polysaccharide derivative; weight ratio of compound a) to compound c) is comprised between 3:1 and 50:1.

The present invention also concerns a hair conditioning and pearlescent system consisting of:

a) an amphoteric surfactant carrying a C₁₆-C₂₀-alkyl or C₁₆-C₂₀-alkenyl;
b) an alcohol compound carrying a C₁₀-C₂₂-alkyl or C₁₀-C₂₂-alkenyl and/or a carboxylic acid compound carrying a C₁₀-C₂₂-alkyl or C₁₀-C₂₂-alkenyl; weight ratio of compound a) to compound b) is comprised between 1:2 and 20:1;
c) a cationic polysaccharide or cationic polysaccharide derivative; weight ratio of compound a) to compound c) is comprised between 3:1 and 50:1.

The present invention also concerns a method for treating a hair in which said hair conditioning and pearlescent composition or said hair conditioning and pearlescent system is used.

The present invention furthermore concerns the use of compounds a), b) and c) to improve the pearlescent effect of a hair conditioning composition.

These and other objects will become readily apparent from the detailed description which follows. The essential as well as optional components of the present compositions are described below.

### DETAILS OF THE INVENTION

Throughout the description, including the claims, the term “comprising one” should be understood as being synonymous with the term “comprising at least one”, unless otherwise specified, and “between” should be understood as being inclusive of the limits.

“Pearlescent” or “nacreous” as used herein means an effect having a pearly luster or gloss and notably providing an iridescent luster resembling that of pearl or mother-of-pearl.

In the context of this invention, “conditioning” is to be understood for purposes of this invention as the brightening treatment of hairs. Conditioning imparts positive properties to the hair, for example improved softness, enhanced shine and color brilliance, a fresh scent, and a decrease in creasing and static charge.

“Alkyl” as used herein means a straight chain or branched saturated aliphatic hydrocarbon group. “Alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbon atoms of the alkenyl group.

The term amphoteric surfactants or zwitterionic surfactants are well-known to the person skilled in the art. It refers to surfactants which, depending on the pH, have anionic and/or cationic properties. They also have an isoelectric point at which they possess a zwitterionic character. In particular, the term refers to compounds having an N⁺ function in combination with an O⁻, C(O)OH, C(O)O⁻, SO₃H or SO₃⁻ function and to compounds having an N function in combination with a C(O)OH, C(O)O⁻, SO₃H or SO₃⁻ function. More in particular, it refers to compounds having an N⁺—O⁻ function, a quaternary N⁺ function in combination with a C(O)O⁻, SO₃H or SO₃⁻ function, and to compounds having a tertiary N function in combination with a C(O)OH, C(O)O⁻, SO₃H or SO₃⁻ function.

For an overview of amphoteric surfactants and their properties the reader is referred to Amphoteric Surfactants, 2^{nd} ed., E. G. Lomax, Ed., 1996, Marcel Dekker. This class of surfactants includes betaines, e.g., fatty alkyl betaines, fatty alkylamido betaines, sulfobetaines, hydroxysulfobetaines, and betaines derived from imidazolines; amine oxides, e.g., fatty alkylamine oxides and fatty alkylamido amine oxides; amphoglycinates and amphopropionates; and so-called “balanced” amphopoly-carboxyglycinates and amphopolycarboxypropionates.

Amphoteric surfactants are exemplified by those which can be broadly described as derivatives of aliphatic quaternary ammonium, phosphonium, and sulfonium compounds, in which the aliphatic radicals can be straight or branched chain, and wherein one of the aliphatic substituents contains a C₁₂-C₂₂-alkyl or C₁₂-C₂₂-alkenyl group, preferably, a C₁₆-C₂₀-alkyl or C₁₆-C₂₀-alkenyl group.

The amphoteric surfactants of the present invention are chosen in the group consisting of: betaine, amine oxide, amphoglycinate and amphopropionate.

Betaines are a class of amphoteric surfactants which include compounds having the structures:

R¹R²R³N⁺(CH₂)_{y}C(O)O⁻ (I)

or

(R¹)(R²C(O)NH(CH₂)ₓ)(R³)N⁺(CH₂)_{y}C(O)O⁻ (II)

wherein R¹ is a C₁-C₅ group which is optionally hydroxylated, such as a methyl, ethyl, hydroxyethyl, or hydroxypropyl group, R² is C₁₂-C₂₂-alkyl or C₁₂-C₂₂-alkenyl group, R³ is independently selected from a C₁-C₅ group or C₁₂-C₂₂-alkyl or C₁₂-C₂₂-alkenyl group as defined for R¹ and R², respectively, x is 2-4, and y is 2-4, and wherein any two of the groups R¹-R³ optionally may form a ring structure. C₁-C₅ group may be an alkyl or alkenyl group. Preferably, R² is C₁₆-C₂₀-alkyl or C₁₆-C₂₀-alkenyl. Preferably, R³ is independently selected from a C₁-C₅ group or C₁₆-C₂₀-alkyl or C₁₆-C₂₀-alkenyl group.

In the context of the present invention, betaine also include sulfobetaines and hydroxysulfobetaines which have structures according to (I) and (II), having R¹, R², and R³ defined as above, wherein the group (CH₂)_{y}C(O)O⁻ has been replaced by a C₃₋₄—SO₃⁻ group, which C₃-C₄ group is optionally hydroxylated.

Amine oxides are a class of amphoteric surfactants which include compounds having the structure:

R¹R²R³N⁺—O⁻ (III)

or

(R¹)(R²C(O)NH(CH²)ₓ)(R³)N⁺—O⁻ (IV)

wherein R¹, R², and R³ and x have the meaning described above.

Amphoglycinates (z=1) and amphopropionates (z=2) are a class of amphoteric surfactants which include compounds having the structures:

R²N(R⁴)(CH₂)_{z}C(O)O⁻Y⁺ (V)

and

R²C(O)N(R⁴)(CH₂)xN(R⁵)(CH₂)_{z}C(O)O⁻Y⁺ (VI)

wherein R² and x have the meaning described above, R⁴ is hydrogen or a C₁-C₅ group which is optionally hydroxylated, R⁵ is a C₁-C₅ group which is optionally hydroxylated or a (CH₂)_{z}C(O)O⁻ group, z is 1-4, and Y⁺ is a cation, such as a proton or a sodium ion.

More preferably, the amphoteric surfactant of the present invention is a compound of formula (VII):

R²—N⁺(CH₃)₂—CH₂—COO⁻ (VII)

wherein R² is C₁₂-C₂₂-alkyl or C₁₂-C₂₂-alkenyl group.

Preferably R² is C₁₆-C₂₂-alkyl, such as C₁₆-alkyl, C₁₈-alkyl, C₂₀-alkyl and C₂₂-alkyl. Preferably R² may also be C₁₆-C₂₂-alkenyl, such as C₁₆-alkenyl, C₁₈-alkenyl, C₂₀-alkenyl and C₂₂-alkenyl.

More preferably, R² is C₁₆-C₂₀-alkyl, such as C₁₆-alkyl, C₁₈-alkyl, and C₂₀-alkyl or C₁₆-C₂₀-alkenyl, such as C₁₆-alkenyl, C₁₈-alkenyl and C₂₀-alkenyl.

In a preferred embodiment of the present invention, the compound of formula (VII) is chosen in the group constituted of: cetyl betaine, palmityl betaine, stearyl betaine and oleyl betaine. More preferably, the compound of formula (VII) is cetyl betaine.

It has to be outlined that the composition of the present invention may comprise an alcohol compound carrying a C₁₀-C₂₂-alkyl or C₁₀-C₂₂-alkenyl and/or a carboxylic acid compound carrying a C₁₀-C₂₂-alkyl or C₁₀-C₂₂-alkenyl.

Preferably, the C₁₀-C₂₂-alkyl or C₁₀-C₂₂-alkenyl carboxylic acids are chosen in the group consisting of: capric acid, lauric acid, myristic acid, palmitic acid, stearic acid, arachidic acid and behenic acid.

Preferably, the C₁₀-C₂₂-alkyl or C₁₀-C₂₂-alkenyl alcohols are chosen in the group consisting of: capryl alcohol, lauryl alcohol, myrityl alcohol, palmityl alcohol, stearyl alcohol, arachidyl alcohol and behenyl alcohol.

Weight ratio of compound a) to compound b) may be comprised between 1:2 and 20:1, notably between 1:1 and 10:1, notably between 1:1 and 5:1.

The composition also comprises at least a cationic polysaccharide or a cationic polysaccharide derivative (also referred to as “compound c)”). Compound c) is preferably chosen in the group consisting of: cationic guar, cationic cellulose, cationic callose, cationic xylan, cationic mannan, cationic galactomannan, and derivatives thereof.

Guars are polysaccharides composed of the sugars galactose and mannose. The backbone is a linear chain of β 1,4-linked mannose residues to which galactose residues are 1,6-linked at every second mannose, forming short side-branches.

Cationic polysaccharides may include cationic polysaccharides that may be obtained by the use of different possible cationic etherifying agents, such as for example the family of quaternary ammonium salts.

In the case of cationic polysaccharides such as cationic guars, the cationic group may be then a quaternary ammonium group bearing 3 radicals, which may be identical or different, preferably chosen from hydrogen, alkyl, hydroxyalkyl, epoxyalkyl, alkenyl, or aryl, preferably containing 1 to 22 carbon atoms, more particularly 1 to 14 and advantageously 1 to 3 carbon atoms. The counterion is generally a halogen, which is one embodiment is chlorine.

Quaternary ammonium salts may be for example: 3-chloro-2-hydroxypropyl trimethyl ammonium chloride (CHPTMAC), 2,3-epoxypropyl trimethyl ammonium chloride (EPTAC), diallyldimethyl ammonium chloride (DMDAAC), vinylbenzene trimethyl ammonium chloride, trimethylammonium ethyl metacrylate chloride, methacrylamidopropyltrimethyl ammonium chloride (MAPTAC), and tetraalkylammonium chloride.

A typical cationic functional group in these cationic polysaccharides is trimethylamino(2-hydroxyl)propyl, with a counter ion. Various counter ions can be utilized, including but not limited to halides, such as chloride, fluoride, bromide, and iodide, sulfate, methylsulfate, and mixtures thereof.

Cationic guars of the present invention may be chosen in the group consisting of:
cationic hydroxyalkyl guars, such as cationic hydroxyethyl guar (HE guar), cationic hydroxypropyl guar (HP guar), cationic hydroxybutyl guar (HB guar), and
cationic carboxylalkyl guars including cationic carboxymethyl guar (CM guar), cationic alkylcarboxy guars such as cationic carboxylpropyl guar (CP guar) and cationic carboxybutyl guar (CB guar), carboxymethylhydroxypropyl guar (CMHP guar).

More preferably, cationic guars of the invention are guars hydroxypropyltrimonium chloride or hydroxypropyl guar hydroxypropyltrimonium chloride.

The degree of hydroxyalkylation (molar substitution or MS) of cationic guars, that is the number of alkylene oxide molecules consumed by the number of free hydroxyl functions present on the guar, may be comprised between 0 and 3, preferably between 0 and 1.7. As example, a MS of 1 may represent one ethylene oxide unit per monosaccharide unit.

The Degree of Substitution (DS) of cationic guars, that is the average number of hydroxyl groups that have been substituted by a cationic group per hydroxyl group per sugar, may be comprised between 0.005 and 1, preferably between 0.01 and 1. DS may notably represent the number of the carboxymethyl groups per monosaccharide unit. DS may notably be determined by titration.

The Charge Density (CD) of cationic guars may be comprised between 0.1 and 2 meq/g, preferably between 0.4 and 1 meq/g. The charge density refers to the ratio of the number of positive charges on a monomeric unit of which a polymer is comprised to the molecular weight of said monomeric unit. The charge density multiplied by the polymer molecular weight determines the number of positively charged sites on a given polymer chain.

The cationic polysaccharides may have an average Molecular Weight (Mw) of between about 100,000 daltons and 3,500,000 daltons, preferably between about 500,000 daltons and 3,500,000 daltons.

Weight ratio of compound a) to compound c) may be comprised between 3:1 and 50:1, preferably between 5:1 and 40:1, more preferably between 10:1 and 30:1.

Composition of the present invention may comprise between 0.1 and 1% by weight of compound c), in relation with the total weight of the composition.

Preferably, the softening composition is substantially free or completely free of anionic agent. Addition of anionic agent(s) may lead to foam forming which is not desired for the present invention, and may negatively affect the conditioning performance of the composition. As used herein, the term “substantially free” when used with reference to the absence of anionic agent in the composition of the present invention, means that the composition comprises less than 0.1 wt % of the anionic agent, more preferably less than 0.01 wt % of the anionic agent, based on the total weight of the composition. As used herein, the term “completely free” when used with reference to the absence of the anionic agent (i.e. 0 wt % of the anionic agent) in the composition of the present invention, means that the composition comprises no anionic agent at all.

The composition of the present invention may notably comprise at least:

a) 1-10% wt of an amphoteric surfactant of formula (VII);
b) 0.1-5% wt of an alcohol compound carrying a C₁₀-C₂₂-alkyl or C₁₀-C₂₂-alkenyl and/or a carboxylic acid compound carrying a C₁₀-C₂₂-alkyl or C₁₀-C₂₂-alkenyl; weight ratio of compound a) to compound b) is comprised between 1:2 and 20:1;
c) 0.01-2% wt of a cationic polysaccharide or cationic polysaccharide derivative; weight ratio of compound a) to compound c) is comprised between 3:1 and 50:1;
d) water.

Preferably, the composition of the present invention may notably comprise at least:

a) 1-10% wt of an amphoteric surfactant of formula (VII);
b) 0.1-5% wt of an alcohol compound carrying a C₁₀-C₂₂-alkyl or C₁₀-C₂₂-alkenyl and/or a carboxylic acid compound carrying a C₁₀-C₂₂-alkyl or C₁₀-C₂₂-alkenyl; weight ratio of compound a) to compound b) is comprised between 1:2 and 20:1;
c) 0.01-2% wt of a cationic guar or cationic guar derivative; weight ratio of compound a) to compound c) is comprised between 3:1 and 50:1;
d) water;
wherein the composition is substantially free or completely free of anionic agent.

In one embodiment, the composition of the present invention consists of:

a) 1-10% wt of an amphoteric surfactant of formula (VII);
b) 0.1-5% wt of an alcohol compound carrying a C₁₀-C₂₂-alkyl or C₁₀-C₂₂-alkenyl and/or a carboxylic acid compound carrying a C₁₀-C₂₂-alkyl or C₁₀-C₂₂-alkenyl; weight ratio of compound a) to compound b) is comprised between 1:2 and 20:1;
c) 0.01-2% wt of a cationic guar or cationic guar derivative; weight ratio of compound a) to compound c) is comprised between 3:1 and 50:1;
d) water.

In another aspect, there is provided a hair conditioning and pearlescent system consisting of:

a) an amphoteric surfactant carrying a C₁₂-C₂₂-alkyl or C₁₂-C₂₂-alkenyl;
b) an alcohol compound carrying a C₁₀-C₂₂-alkyl or C₁₀-C₂₂-alkenyl and/or a carboxylic acid compound carrying a C₁₀-C₂₂-alkyl or C₁₀-C₂₂-alkenyl; weight ratio of compound a) to compound b) is comprised between 1:2 and 20:1;
c) a cationic polysaccharide or cationic polysaccharide derivative; weight ratio of compound a) to compound c) is comprised between 3:1 and 50:1.

In one embodiment, there is provided a hair conditioning and pearlescent system consisting of:

a) an amphoteric surfactant carrying a C₁₆-C₂₀-alkyl or C₁₆-C₂₀-alkenyl;
b) an alcohol compound carrying a C₁₀-C₂₂-alkyl or C₁₀-C₂₂-alkenyl and/or a carboxylic acid compound carrying a C₁₀-C₂₂-alkyl or C₁₀-C₂₂-alkenyl; weight ratio of compound a) to compound b) is comprised between 1:2 and 20:1;
c) a cationic polysaccharide or cationic polysaccharide derivative; weight ratio of compound a) to compound c) is comprised between 3:1 and 50:1.

The hair conditioning and pearlescent system is preferably mixed with a suitable liquid carrier which may be selected from water, organic solvents and mixtures thereof.

For optimum phase stability of these compositions, the neat pH, measured at 20° C., may be in the range of from 3 to 8. The pH of these compositions herein can be regulated by the addition of acids such as Bronsted or Lewis ones. Examples of suitable acids include the inorganic mineral acids, carboxylic acids, in particular the low molecular weight (C₁-C₅)-carboxylic acids, and alkylsulfonic acids. Suitable inorganic acids include HCl, H₂SO₄, HNO₃ and H₃PO₄. Suitable organic acids include formic, acetic, citric, methylsulfonic and ethylsulfonic acid. Preferred acids are citric, hydrochloric, phosphoric, formic, methylsulfonic acid, and benzoic acids. Especially preferred is citric acid.

The compositions of the present invention may be manufactured using conventional formulation and mixing techniques. It is notably possible to simultaneously or sequentially blend components a), b) and/or c). In one procedure for manufacture, components a), b) and c) are premixed prior to the addition of the remaining components.

For instance, compositions may notably be obtained as follows:
Mix component a), b) and c) into water till it is homogeneous
Heat the above mixture to 80° C. under stirring
Cool down to room temperature and discharge

Preferably the compositions are obtained as follows:
Mix component c) into water till it is homogeneous
Add component a) into the above mixture and heat up to 80° C.
Heat component b) separately to 80° C.
Mix above components altogether at 80° C. and keep agitation
Cool down to room temperature and discharge.

Methods of making compositions of the present invention are described more specifically in the following experimental part.

The compositions of the present invention may also comprise other components such as organic or inorganic thickeners such as hydroxyethyl cellulose, opacifying agents such as for example ethyleneglycol distearate, water-insoluble skin benefit agents, exfoliating particles, preservatives, antimicrobials, bactericides, antioxydants such as butylated hydroxytoluene, humectants, fragrances, colouring agents, cosmetic fillers, herb and plant extracts, and sequestering agents such as for example sodium salt of the ethylenediaminetetraacetic acid.

The humectants in the composition may be selected from glycerine, diols, triols and polyols. The composition may comprise from 0 to 10% by weight of humectants, based on the total weight of the composition.

Preferably, the composition of the present invention is substantially free or, in some cases, completely free of any quaternary ammonium salts except the compounds of a), b) and c). Quaternary means that the ammonium (N) has four chemical groups attached, such as alkyl or alkenyl groups. As used herein, the term “substantially free” when used with reference to the absence of quaternary ammonium salts in the medium of the present invention, means that the composition comprises less than 0.1 wt %, preferably less than 0.01 wt %, based on the total weight of the composition. As used herein, the term “completely free” when used with reference to the absence of quaternary ammonium salts in the composition, means that the composition comprises no quaternary ammonium salts at all. These notably include cetrimonium chloride (CTAC), behentrimonium chloride (BTAC), di(palmiticcarboxyethyl) hydroxyethyl methyl ammonium methylsulfate (TEP), and dimethyl di(hydrogenated tallow) ammonium chloride (DHT), N,N-bis(stearoyl-oxy-ethyl) N,N-dimethyl ammonium chloride, N,N-bis(tallowoyl-oxy-ethyl) N,N-dimethyl ammonium chloride, N,N-bis(stearoyl-oxy-ethyl) N-(2-hydroxyethyl) N-methyl ammonium methylsulfate and 1,2-di(stearoyl-oxy)-3-trimethyl ammoniumpropane chloride.

The composition of the present invention preferably comprises less than 1 wt % of pearlescent agent except the compounds of a), b) and c).

Preferably, the composition of the present invention is substantially free or, in some cases, completely free of any pearlescent agent except the compounds of a), b) and c). As used herein, the term “substantially free” when used with reference to the absence of pearlescent agent in the medium of the present invention, means that the composition comprises less than 0.5 wt %, preferably less than 0.1 wt %, based on the total weight of the composition. As used herein, the term “completely free” when used with reference to the absence of pearlescent agent in the composition, means that the composition comprises no pearlescent agent at all.

The composition according to the present invention, may take a variety of physical forms including liquid, liquid-gel, paste-like, foam in either aqueous or non-aqueous form, powder, granular and tablet forms. For better dispersability, a preferred form of the composition is a liquid form, and in the form of an aqueous dispersion in water. When in a liquid form, the composition may also be dispensed with dispensing means such as a sprayer or aerosol dispenser.

When in a liquid form, such a composition may contain from 0.1% to 20% by weight of compounds a), b) and c). The composition will usually also contain water and other additives, which may provide the balance of the composition. Suitable liquid carriers are selected from water, organic solvents and mixtures thereof. The liquid carrier employed in the instant compositions is preferably at least primarily water due to its low cost, safety, and environmental compatibility. Mixtures of water and organic solvent may be used. Preferred organic solvents are; monohydric alcohol, such as ethanol, propanol, iso-propanol or butanol; dihydric alcohol, such as glycol; trihydric alcohols, such as glycerol, and polyhydric (polyol) alcohols.

These compositions of the invention may be formulated for washing skin and/or hair, for example, bath or shower gels, handwashing compositions, facial washing compositions, pre- and post-shaving products, and rinse-off and wipe-off skin care products, and mainly to produce cleansing foam, body shampoo and hair shampoo.

Compositions of the invention are used in a manner known for leave-in and wash-out conditioners. Namely, the hair is wet and then conditioner or conditioning shampoo is applied to the hair. If the conditioner is a leave-in conditioner, it can be applied to wet or dry hair. If applied to dry hair, then water is added after such application. If the conditioner is a wash-out conditioner, the hair is the rinsed after application.

The present invention also provides methods for conditioning hair. The present compositions are used in conventional ways to provide the optimized hair conditioning benefits of the present invention. Such methods generally involve application of an effective amount of the conditioning composition to the hair, which is massaged through and then rinsed from the hair. By “effective amount” is meant an amount sufficient to provide the desired conditioning benefits considering the length, texture and condition of the hair. The present invention also provides methods for conditioning hair comprising the step of contacting the hair with an aqueous medium comprising the hair conditioning and pearlescent system as described above.

After the hair is treated with the present conditioning compositions the hair is dried and styled in the usual ways of the user. The hair conditioning compositions provide optimized hair conditioning benefits, such as soft wet and dry hair feel, ease of wet and dry hair combing and hair detangling, increased hair manageability, and anti-static benefits.

The present invention also provides the use of the hair conditioning and pearlescent composition described above for improving the pearlescent appearance of the hairs.

Should the disclosure of any patents, patent applications, and publications which are incorporated herein by reference conflict with the description of the present application to the extent that it may render a term unclear, the present description shall take precedence.

The examples provided here further describe and demonstrate embodiments of the present invention. The examples are given solely for the purpose of illustration and are not to be construed as limitation of the present invention.

### EXPERIMENTAL PART

All parts, percentages, and ratios herein are by weight unless otherwise specified.

The following are components of the hair conditioning compositions:
CAPB: Cocamidopropyl betaine
CB: cetyl betaine
Tween20: PEG20 sorbitan laurate
PA: palmityl alcohol
EGDS: ethylene glycol di stearate
Guar 1: guar hydroxypropyltrimonium chloride with a Mw of 2 M, a degree of substitution (DS) of 0.10-0.13 and a charge density of 0.6-0.7 meq/g
Guar 2: natural guar gum providing an average Mw of 2.5 M

### Example 1

### Formulations

1. In a side beaker, heat the D.I. water up to 80° C.
2. In the main beaker, add compounds as mentioned in Tables in water, use IKA mechanical overhead stirrer to stir at 50 rpm while heating up to 80° C. When the mixture is melt and becomes clear, pour the pre-heated D.I. water in Step 1 into the main beaker, maintain the temperature of 80° C., stir at 1000 rpm for 2 minutes.
3. Remove heat, stir at 400 rpm to cool down the system.
4. When temperature is below 45° C., add Mackstat DM, stir for another 15 minutes, stop stirring and cool down to room temperature.
5. Discharge.

All of the formulations comprise 0.4% by weight of fragrances and preservatives.

### Example 2

### Conditioning Effect Measurement (Dia-Stron Wet/Dry Combing)

The changes in the wet and/or dry combing attributes of hair resulting from treatment with shampoos, conditioners, styling aids or other active materials were measure using DIA-STRON MTT-170 (Miniature Tensile Tester) as follows.

### Materials and Equipment:

Dia-Stron MTT 170
Dia-Stron software “UVWin 1.29.4000”
IBM compatible computer and printer
Hair tresses (2 grams, 6 inches long, net 1 inch wide, soft swatch—International Hair Importers, N.Y.)
Coarse comb (4-tine/cm) for combing hair
Fine comb (6-tine/cm) to be attached on Dia-Stron fixed arm
Standard rinsing apparatus: tray filled with running water (temp˜25-40° C.; flow-rate˜1.8 L/min)

Experimental conditions: with the exception of pre-treatment and treatment of hair tresses, all procedures should be conducted in a humidity and temperature-controlled room (60% relative humidity, 21−22° C.)

Procedures to be carried out:

1. Pre-treatment of hair tresses/To remove residual conditioning agents and other impurities
2. Wet combing (sorting)/For sorting of hair tresses
3. Dry combing (untreated)/Base line of dry, untreated tresses
4. Wet combing (untreated)/Base line of wet, untreated tresses
5. Treatment of hair tresses with test shampoo, conditioners, styling aids or other active materials formulations
6. Dry combing (treated)/Effect of treatment and conditioning
7. Wet combing (treated)/Effect of treatment and conditioning

### *Note:

1 hair tress=10 combings
1 test sample=3 hair tresses
3 hair tresses×10 combings=30 combings per test sample

### Data Processing:

Conditioning Effect (Total Work Reduction)=(Treated Dry−Untreated Dry)/Untreated Dry %

Conditioning Effect (Total Work Reduction)=(Treated Wet−Untreated Wet)/Untreated Wet %

Conditioning Effect (Greatest Force)=(Treated Dry−Untreated Dry)/Untreated Dry %

Conditioning Effect (Greatest Force)=(Treated Wet−Untreated Wet)/Untreated Wet %

Results are expressed in Tables 1 and 2 as follows.

TABLE 1
Formulations of the invention

| Formulations | F1 | F2 | F3 |
|---|---|---|---|
| Surfactants | CB | CB | CB |
| (% by wt) | (2.1) | (2.1) | (2.1) |
| Acid, ester | PA | PA | PA |
| or alcohol | (1.4) | (1.4) | (1.4) |
| (% by wt) | | | |
| Polysaccharide | Guar 1 | Guar 1 | Guar 1 |
| (% by wt) | (0.1) | (0.5) | (0.3) |
| Properties | | | |
| Wet combing | −52 | −77 | −87.6 |
| Change in | | | |
| Peak Load (%) | | | |
| Wet combing | −56 | −79 | −81 |
| Change in | | | |
| Total Work (%) | | | |
| Dry combing | −88 | −93 | −92.4 |
| Change in | | | |
| Peak Load (%) | | | |
| Dry combing | −67 | −84 | −79.3 |
| Change in | | | |
| Total Work (%) | | | |
| Pearlescent | 1.5 | 1.5 | 1.5 |
| properties | | | |

TABLE 2
Formulations of comparison

| Formulations | C1 | C2 | C3 | C4 | C5 | C6 |
|---|---|---|---|---|---|---|
| Surfactants | CB | CB | CB | Tween20 | CB | CAPB |
| (% by wt) | (3) | (3) | (2.1) | (2.1) | (2.1) | (2.1) |
| Acid, ester | — | PA | PA | PA | EGDS | PA |
| or alcohol | | (2) | (1.4) | (1.4) | (1.4) | (1.4) |
| (% by wt) | | | | | | |
| Polysaccharide | — | — | Guar 2 | Guar 1 | Guar 1 | Guar 1 |
| (% by wt) | | | (0.3) | (0.3) | (0.1) | (0.3) |
| Properties | | | | | | |
| Wet combing | +11.1 | −3 | +12.8 | −59 | −38 | −12.5 |
| Change in | | | | | | |
| Peak Load (%) | | | | | | |
| Wet combing | −18 | −18 | −5.6 | −67 | −62 | −17.1 |
| Change in Total | | | | | | |
| Work (%) | | | | | | |
| Dry combing | −73.9 | −84 | −65.3 | +278 | −88 | −45.3 |
| Change in | | | | | | |
| Peak Load (%) | | | | | | |
| Dry combing | −50.3 | −75 | −43.6 | +152 | −80 | −42.1 |
| Change in Total | | | | | | |
| Work (%) | | | | | | |
| Pearlescent | 0 | 2 | 1.5 | 0 | 0 | 0 |
| properties | | | | | | |

The pearlescent effect is evaluated by a 10 people panel team with the naked eye at a temperature of 25° C. Double-mind assessment is made with 10 ml of composition dropped on a black screen. Incidentally, a composition in which bubbles are commingled is centrifuged to remove bubbles. A 3 grades system is used for the results: 0 means no pearlescent effect [no luster]; 1 means some pearlescent effect [low luster] and 2 means very good [high luster].

It appears then that the formulations of the present invention provide a good pearlescent appearance and conditioning effect for wet and dry hair. By contrast, formulations of comparison provide either low conditioning properties on wet hair and/or dry hair or no good pearlescent effect. Notably, the composition comprising CB (C₁₆) provided better conditioning performance and pearlescent effect in comparison of that comprising CAPB (C₁₂).

1-21: (canceled)

22. A hair conditioning and pearlescent composition comprising at least:
a) 1-10% wt of an amphoteric surfactant carrying a C₁₂-C₂₂-alkyl or C₁₂-C₂₂-alkenyl;
b) 0.1-5% wt of an alcohol compound carrying a C₁₀-C₂₂-alkyl or C₁₀-C₂₂-alkenyl and/or a carboxylic acid compound carrying a C₁₀-C₂₂-alkyl or C₁₀-C₂₂-alkenyl; weight ratio of compound a) to compound b) is comprised between 1:2 and 20:1;
c) 0.01-2% wt of a cationic polysaccharide or cationic polysaccharide derivative; weight ratio of compound a) to compound c) is comprised between 3:1 and 50:1; and
d) water;
wherein the percent by weight is expressed in relation with the total weight of the composition, and the composition comprises less than 0.5 wt % of quaternary ammonium salts, except compounds a), b), and c).

23. The hair conditioning and pearlescent composition according to claim 22, wherein the amphoteric surfactant carries a C₁₆-C₂₂-alkyl or C₁₆-C₂₂-alkenyl.

24. The hair conditioning and pearlescent composition according to claim 22, wherein the amphoteric surfactant carries a C₁₆-C₂₀-alkyl or C₁₆-C₂₀-alkenyl group.

25. The hair conditioning and pearlescent composition according to claim 22, wherein the hair conditioning and pearlescent composition is substantially free or completely free of anionic agent.

26. The hair conditioning and pearlescent composition according to claim 22, wherein the amphoteric surfactant is selected from the group consisting of: betaine, amine oxide, amphoglycinate, and amphopropionate.

27. The hair conditioning and pearlescent composition according to claim 26, wherein the betaine has the structure:
R¹R²R³N⁺(CH₂)_{y}(C(O)O⁻ (I)
or
(R¹)(R²C(O)NH(CH₂)ₓ)(R³)N⁺CH₂)_{y}C(O)O⁻ (II)
wherein R¹ is a C₁-C₅ group which is optionally hydroxylated, R² is C₁₆-C₂₀-alkyl or C₁₆-C₂₀-alkenyl group, R³ is independently selected from a C₁-C₅ group or C₁₆-C₂₀-alkyl or C₁₆-C₂₀-alkenyl group, respectively, x is 2-4, and y is 2-4, and wherein any two of the groups R¹-R³ optionally form a ring structure.

28. The hair conditioning and pearlescent composition according to claim 26, wherein the amine oxide has the structure:
R¹R²R³N⁺—O⁻ (III)
or
(R¹)(R²C(O)NH(CH²)ₓ)(R³)N⁺—O⁻ (IV)
wherein R¹ is a C₁-C₅ group which is optionally hydroxylated, R² is C₁₆-C₂₀-alkyl or C₁₆-C₂₀-alkenyl group, R³ is independently selected from a C₁-C₅ group or C₁₆-C₂₀-alkyl or C₁₆-C₂₀-alkenyl group, respectively, x is 2-4, and y is 2-4, and wherein any two of the groups R¹—R³ optionally form a ring structure.

29. The hair conditioning and pearlescent composition according to claim 26, wherein the amphoglycinate (z=1) and amphopropionate (z=2) have the structures:
R²N(R⁴)(CH₂)_{z}C(O)O⁻Y⁺ (V)
and
R²C(O)N(R⁴)(CH₂)xN(R⁵)(CH₂)_{z}C(O)O⁻Y⁺ (VI)
wherein R² is C₁₆-C₂₀-alkyl or C₁₆-C₂₀-alkenyl group, x is 2-4, and y is 2-4, R⁴ is hydrogen or a C₁-C₅ group which is optionally hydroxylated, R⁵ is a C₁-C₅ group which is optionally hydroxylated or a (CH₂), C(O)O⁻ group, z is 1-4, and Y⁺ is a cation.

30. The hair conditioning and pearlescent composition according to claim 22, wherein the amphoteric surfactant has the formula (VII):
R²—N⁺(CH₃)₂—CH₂—COO⁻ (VII)
wherein R² is C₁₆-C₂₀-alkyl or C₁₆-C₂₀-alkenyl group.

31. The hair conditioning and pearlescent composition according to claim 30, wherein the compound of formula (VII) is selected from the group consisting of: cetyl betaine, palmityl betaine, stearyl betaine, and oleyl betaine.

32. The hair conditioning and pearlescent composition according to claim 22, wherein the C₁₀-C₂₂-alkyl or C₁₀-C₂₂-alkenyl carboxylic acids are selected from the group consisting of: capric acid, lauric acid, myristic acid, palmitic acid, stearic acid, arachidic acid, and behenic acid.

33. The hair conditioning and pearlescent composition according to claim 22, wherein the C₁₀-C₂₂-alkyl or C₁₀-C₂₂-alkenyl alcohols are selected from the group consisting of: capryl alcohol, lauryl alcohol, myrityl alcohol, palmityl alcohol, stearyl alcohol, arachidyl alcohol, and behenyl alcohol.

34. The hair conditioning and pearlescent composition according to claim 22, wherein the compound c) is selected from the group consisting of: cationic guar, cationic cellulose, cationic callose, cationic xylan, cationic mannan, cationic galactomannan, and derivatives thereof.

35. The hair conditioning and pearlescent composition according to claim 22, wherein the compound c) is guar hydroxypropyltrimonium chloride or hydroxypropyl guar hydroxypropyltrimonium chloride.

36. The hair conditioning and pearlescent composition according to claim 22, wherein the hair conditioning and pearlescent composition comprises less than 0.01 wt % of the quaternary ammonium salt.

37. The hair conditioning and pearlescent composition according to claim 22, wherein the composition comprises less than 1 wt % of pearlescent agent, except compounds a), b) and c).

38. A hair conditioning and pearlescent system consisting of:
a) an amphoteric surfactant carrying a C₁₂-C₂₂-alkyl or C₁₂-C₂₂-alkenyl;
b) an alcohol compound carrying a C₁₀-C₂₂-alkyl or C₁₀-C₂₂-alkenyl and/or a carboxylic acid compound carrying a C₁₀-C₂₂-alkyl or C₁₀-C₂₂-alkenyl; weight ratio of compound a) to compound b) is comprised between 1:2 and 20:1; and
c) a cationic polysaccharide or cationic polysaccharide derivative; weight ratio of compound a) to compound c) is comprised between 3:1 and 50:1.

39. The hair conditioning and pearlescent system according to claim 38, wherein the amphoteric surfactant carries a C₁₆-C₂₀-alkyl or C₁₆-C₂₀-alkenyl.

40. A method for treating hair, wherein the hair conditioning and pearlescent composition according to claim 22 is contacted with the hair.

41. A method for treating hair comprising the step of contact the hair with an aqueous medium comprising the hair conditioning and pearlescent system according to claim 38.

42. The method according to claim 41, wherein the pearlescent appearance of the hair is improved after contacting the hair with the aqueous medium comprising the hair conditioning and pearlescent system.
